(19) United States
(12) Patent Application Publication
(10) Pub. No.: US 2025/0226113 A1
(43) Pub. Date: Jul. 10, 2025

(54) DEVICE FOR OUTPUTTING SIMULATED VITAL SIGN AND OPERATION METHOD THEREOF

(71) Applicants: Samsung Electronics Co., Ltd., Suwon-si (KR); Samsung Electronics Co., Ltd., Suwon-si (KR)

(72) Inventor: Andrii OMELCHENKO, Kyiv (UA)

(73) Assignee: Samsung Electronics Co., Ltd., Suwon-si (KR)

(21) Appl. No.: 19/088,529

(22) Filed: Mar. 24, 2025

Related U.S. Application Data
(63) Continuation of application No. PCT/KR2023/011242, filed on Aug. 1, 2023.

(30) Foreign Application Priority Data
Sep. 23, 2022 (KR) 10-2022-0121131

(51) Int. Cl. G16H 50/50; A61B 5/05; G01S 13/02; G06F 21/62; G16H 50/30

(52) U.S. Cl. CPC G16H 50/50; A61B 5/05; G01S 13/0209; G06F 21/6245; G16H 50/30

(57) ABSTRACT

A device for outputting a simulated vital sign, includes: an ultra-wideband (UWB) communication interface including a UWB radar and a UWB antenna, and configured to detect a UWB scanning pulse transmitted by an external device to measure a vital sign of a user in a non-contact manner; memory storing at least one instruction; and at least one processor configured to execute the at least one instruction, wherein the at least one instruction, when executed the at least one processor individually or collectively, cause the device to: based on the UWB scanning pulse being detected by the UWB communication interface, generate a virtual vital sign by simulating a vital sign of a virtual person, obtain simulated vital sign data by combining the vital sign with the generated virtual vital sign, and output, through the UWB antenna, a UWB signal component including the vital sign data toward the external device.

### CROSS-REFERENCE TO RELATED APPLICATIONS

This application is a continuation of International Application No. PCT/KR2023/011242, which is based on and claims priority to Korean Patent Application No. 10-2022-0121131, filed on Sep. 23, 2022, in the Korean Intellectual Property Office, the disclosures of which are incorporated by reference herein in their entireties.

### BACKGROUND

### 1. Field

The disclosure relates to a device for outputting a simulated vital sign and an operation method thereof, and more particularly, to a device for outputting simulated vital sign data to an external device in a non-contact manner and an operation method thereof.

### 2. Description of Related Art

Technologies for measuring a user's vital signs by using sensors in devices have become more prevalent. By using vital sign measurement technology using a sensor, it is possible to directly or indirectly estimate a user's current state of health, as well as indirectly estimate a user's current stress level, current context, or reaction to a change in context (e.g., a word, a text message, a photo, or the like). A user's emotional state is a matter of personal privacy, and therefore may be protected through authority granted by the user to a device.

In contact-based vital sign measurement technology using a sensor, privacy may be sufficiently protected by granting authority to a device. However, technology for measuring a user's vital signs (e.g., respiration rate, heart rate, etc.) in a non-contact manner by using a latest mobile device such as a smartphone that includes an ultra-wideband (UWB) radar has been utilized in recent years. Because the non-contact vital sign measurement technology may also be used to measure vital signs of a person other than the user of the device without permission, the privacy (vital signs) of the other person cannot be protected merely by the user granting permission to the device. In order to measure vital signs of another person in a non-contact manner, the other person needs to be asked for permission, but a device of the other person cannot be accessed.

From the other person's perspective, there is a need for a method for preventing measurement of vital signs by an external device in a non-contact manner and protecting privacy.

### SUMMARY

According to an aspect of the disclosure, a device for outputting a simulated vital sign, includes: an ultra-wideband (UWB) communication interface including a UWB radar and a UWB antenna, the UWB communication interface being configured to detect a UWB scanning pulse transmitted by an external device to measure a vital sign of a user in a non-contact manner; at least one processor including processing circuitry; and memory storing one or more instructions; wherein the one or more instructions are configured to, when executed by the at least one processor individually or collectively, cause the device to: based on the UWB scanning pulse being detected by the UWB communication interface, generate a virtual vital sign by simulating a vital sign of a virtual person, obtain simulated vital sign data by combining the vital sign with the generated virtual vital sign, and output, through the UWB antenna, a UWB signal component including the vital sign data toward the external device.

The one or more instructions are configured to, when executed by the at least one processor individually or collectively, further cause the device to: generate the virtual vital sign by performing a simulation using the vital sign and context information.

The context information may include information about at least one of sound, vibration, illumination, or atmospheric pressure of an environment or situation surrounding the device and the external device.

The one or more instructions are configured to, when executed by the at least one processor individually or collectively, further cause the device to: detect a change in the vital sign, and generate the virtual vital sign that compensates for the change in the vital sign through a simulation using the detected change in the vital sign.

The device may further include: a microphone configured to obtain a voice of the user or a sound from an external object; an inertial measurement unit (IMU) sensor configured to measure at least one of an acceleration, an angular velocity, or a gravity direction; and an ambient light sensor configured to measure illuminance of an external environment, and the one or more instructions are configured to, when executed by the at least one processor individually or collectively, further cause the device to: obtain context data about the external environment from at least one of the microphone (140), the IMU sensor, and the ambient light sensor, delay, by a preset time, the vital sign, the virtual vital sign, and the context data, and output, through the UWB antenna, the delayed vital sign, the delayed virtual vital sign, and the delayed context data to the external device.

The one or more instructions are configured to, when executed by the at least one processor individually or collectively, further cause the device to, by delaying the vital sign, the virtual vital sign, and the context data by respectively applying different delay times to the vital sign, the virtual vital sign, and the context data, control a timing at which the delayed vital sign, the delayed virtual vital sign, and the delayed context data are output through the UWB antenna.

The one or more instructions are configured to, when executed by the at least one processor individually or collectively, further cause the device to: obtain a UWB signal component by adding a noise signal to the vital sign data, and output, through the UWB antenna, the generated UWB signal component toward the external device.

According to an aspect of the disclosure, a method, performed by a device, of outputting a simulated vital sign, includes: detecting an ultra-wideband (UWB) scanning pulse transmitted by an external device to measure a vital sign of a user in a non-contact manner; based on the UWB scanning pulse being detected, generating a virtual vital sign by simulating a vital sign of a virtual person; obtaining simulated vital sign data by combining the vital sign with the generated virtual vital sign; and outputting, through a UWB antenna of the device, a UWB signal component including the vital sign data toward the external device.

The generating of the virtual vital sign may include generating the virtual vital sign by performing a simulation using the vital sign and context information.

The context information may include information about at least one of sound, vibration, illumination, or atmospheric pressure of an environment or situation surrounding the device and the external device.

The generating the virtual vital sign may include: detecting a change in the vital sign; and
generating the virtual vital sign that compensates for the change in the vital sign through a simulation using the detected change in the vital sign.

The method may further include: obtaining context data about an external environment by using a sensor, and the outputting the UWB signal component may include delaying, by a preset time, the vital sign, the virtual vital sign, and the context data and outputting the delayed vital sign, the delayed virtual vital sign, and the delayed context data to the external device through the UWB antenna.

The outputting the UWB signal component may include, by delaying the vital sign, the virtual vital sign, and the context data by respectively applying different delay times to the vital sign, the virtual vital sign, and the context data, controlling a timing at which the delayed vital sign, the delayed virtual vital sign, and the delayed context data are output through the UWB antenna.

The outputting the vital sign data toward the external device may include: obtaining a UWB signal component by adding a noise signal to the vital sign data; and outputting the generated UWB signal component toward the external device via the UWB antenna.

According to an aspect of the disclosure, a computer program product includes a non-transitory computer-readable storage medium storing instructions that are executed by at least one processor of a device to perform a method of outputting a simulated vital sign, the method including: detecting an ultra-wideband (UWB) scanning pulse transmitted by an external device to measure a vital sign of a user in a non-contact manner; based on the UWB scanning pulse being detected, generating a virtual vital sign by simulating a vital sign of a virtual person; obtaining simulated vital sign data by combining the vital sign with the generated virtual vital sign; and outputting, through a UWB antenna of the device, a UWB signal component including the vital sign data toward the external device.

### BRIEF DESCRIPTION OF THE DRAWINGS

The above and other aspects, features, and advantages of certain embodiments of the present disclosure will be more apparent from the following description taken in conjunction with the accompanying drawings, in which:

FIG. 1 is a diagram illustrating an operation in which a device measures a user's vital sign in a non-contact manner;

FIG. 2 is a conceptual diagram illustrating an operation in which a device recognizes an ultra-wideband (UWB) scanning pulse transmitted by an external device and outputs simulated vital sign data, according to an embodiment of the present disclosure;

FIG. 3 is a block diagram illustrating components of a device according to an embodiment of the present disclosure;

FIG. 4 is a flowchart of an operation method of a device, according to an embodiment of the present disclosure;

FIG. 5 is a diagram illustrating an operation in which a device obtains simulated vital sign data, according to an embodiment of the present disclosure;

FIG. 6 is a flowchart of a method, performed by a device, of generating a virtual vital sign based on a change in a user's vital sign, according to an embodiment of the present disclosure;

FIG. 7 is a flowchart of a method, performed by a device, of generating a virtual vital sign by performing a simulation for compensating for a change in a user's vital sign, according to an embodiment of the present disclosure;

FIG. 8 is a diagram illustrating simulated vital sign data before and after a change in a vital sign occurs;

FIG. 9 is a diagram illustrating whether an external device is able to distinguish a user's vital sign based on the number of virtual vital signs generated by a device, according to an embodiment of the present disclosure;

FIG. 10 is a flowchart of a method, performed by a device, of delaying a vital sign, a virtual vital sign, and context data, and outputting the delayed vital sign, the delayed virtual vital sign, and the delayed context data to an external device, according to an embodiment of the present disclosure; and

FIG. 11 is a diagram illustrating an operation in which a device delays a vital sign, a virtual vital sign, and context data, and outputs the delayed vital sign, the delayed virtual vital sign, and the delayed context data to an external device, according to an embodiment of the present disclosure.

### DETAILED DESCRIPTION

As terms used in embodiments of the present specification, general terms that are currently widely used are selected by taking into account functions in the present disclosure, but these terms may vary according to the intention of one of ordinary skill in the art, precedent cases, advent of new technologies, etc. Furthermore, specific terms may be arbitrarily selected by the applicant, and in this case, the meaning of the selected terms will be described in detail in the detailed description of a corresponding embodiment. Thus, the terms used herein should be defined not by simple appellations thereof but based on the meaning of the terms together with the overall description of the present disclosure.

Singular expressions used herein are intended to include plural expressions as well unless the context clearly indicates otherwise. All the terms used herein, which include technical or scientific terms, may have the same meaning that is generally understood by a person of ordinary skill in the art described in the present disclosure.

Throughout the present disclosure, the expression “at least one of a, b or c” indicates only a, only b, only c, both a and b, both a and c, both b and c, or all of a, b, and c.

Throughout the present disclosure, when a part “includes” or “comprises” an element, unless there is a particular description contrary thereto, it is understood that the part may further include other elements, not excluding the other elements. Furthermore, terms, such as “ . . . portion” “ . . . module” etc., used herein indicate a unit for processing at least one function or operation, and may be implemented as hardware or software or a combination of hardware and software.

The expression “configured to (or set to)” used herein may be used interchangeably, according to context, with, for example, the expression “suitable for,” “having the capacity to,” “designed to,” “adapted to,” “made to,” or “capable of”. The term “configured to (or set to)” may not necessarily mean only “specifically designed to” in terms of hardware. Instead, the expression “a system configured to” may mean, in some contexts, the system being “capable of”, together with other devices or components. For example, the expression “a processor configured to (or set to) perform A, B, and C” may mean a dedicated processor (e.g., an embedded processor) for performing the corresponding operations, or a general-purpose processor (e.g., a central processing unit (CPU) or an application processor) capable of performing the corresponding operations by executing one or more software programs stored in a memory.

Furthermore, in the present disclosure, when a component is referred to as being “connected” or “coupled” to another component, it should be understood that the component may be directly connected or coupled to the other component, but may also be connected or coupled to the other component via another intervening component therebetween unless there is a particular description contrary thereto.

As used herein, ‘ultra-wideband (UWB) communication’ refers to a communication method that uses an ultra-wideband frequency band between 3.1 gigahertz (GHz) and 10.6 GHz to transmit and receive data. UWB communication networks are capable of transmitting and receiving data at speeds of up to 500 megabits per second (Mbps).

In the present disclosure, a ‘vital sign’ is a signal indicating a user's physical and health status, and may also be referred to as vital sign data’. The vital sign may include, for example, a measurement of a person's body temperature, blood pressure, respiration rate, heart rate, or heartbeat interval.

As used herein, a ‘virtual person’ refers to a fake person virtually created through simulation, rather than a user of a device.

In the present disclosure, a ‘virtual vital sign’ refers to a vital sign of a virtual person. In an embodiment of the present disclosure, a virtual vital sign may be generated through a simulation based on a user's vital sign. However, the present disclosure is not limited thereto. The virtual vital sign may include, for example, at least one of a virtual respiration rate, a virtual heart rate, and a virtual heartbeat interval.

An embodiment of the present disclosure will be described more fully hereinafter with reference to the accompanying drawings so that the embodiment may be easily implemented by a person of ordinary skill in the art. However, the present disclosure may be implemented in different forms and should not be construed as being limited to embodiments set forth herein.

Hereinafter, embodiments of the present disclosure are described in detail with reference to the accompanying drawings.

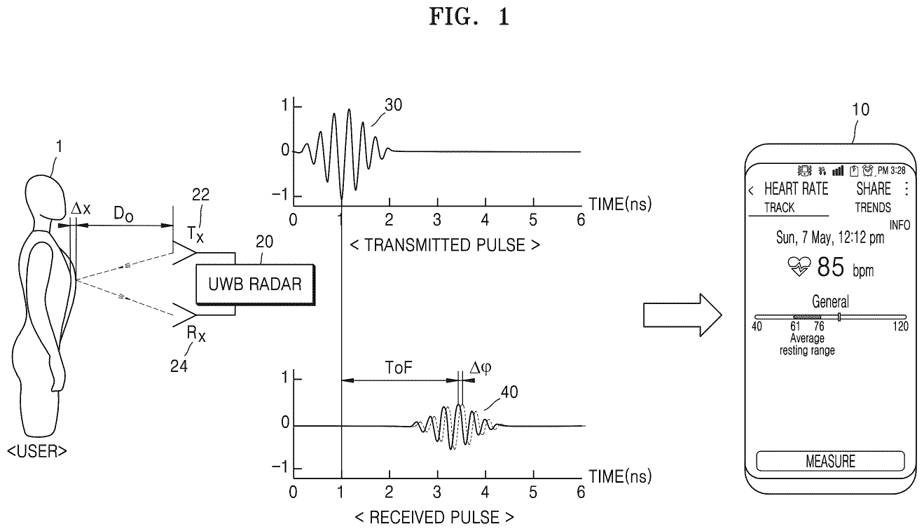

FIG. 1 is a diagram illustrating an operation in which a device 10 measures a vital sign of a user 1 in a non-contact manner.

Referring to FIG. 1, the device 10 may include an ultra-wideband (UWB) radar 20, a transmitting antenna 22, and a receiving antenna 24. The device 10 may generate a UWB impulse signal 30 having a UWB band by using the UWB radar 20, and transmit the UWB impulse signal 30 to a chest of the user 1 via the transmitting antenna 22. The transmitted UWB impulse signal 30 is reflected by a body of the user 1, and the device 10 may receive a reflected UWB impulse signal 30 by using the receiving antenna 24.

When the user 1 breathes, a mechanical displacement Ax between the chest and an abdominal wall occurs, which causes a variation in a time of flight (ToF) that is a time interval between a peak of the transmitted UWB impulse signal 30 and a peak of the received UWB impulse signal 40. The device 10 may amplify and filter the UWB impulse signal 40 received via the receiving antenna 24, and obtain respiration rate information by measuring and analyzing at least one of the number and position of peaks with a predetermined amplitude or greater and an interval between adjacent peaks in a respiratory signal over a predetermined time period in the time domain. In the embodiment illustrated in FIG. 1, the device 10 may filter a received pulse reflected from the body of the user 1 by using a median filter, a Kalman filter, a band pass filter, or the like, and measure a respiration rate based on a distance D₀ between the receiving antenna 24 and the body of the user 1 and a peak interval Δφ that changes due to respiration.

In an embodiment of the present disclosure, the device 10 may extract a peak interval change value Δφ from the received pulse, extract a heart rate for each distance by applying a band pass filter and a frequency axis transform (e.g., a chirp Z-transform (CZT)), and measure the heart rate from a peak value per unit time.

The method of measuring a vital sign including the respiration rate and the heart rate of the user 1 in a non-contact manner by using a UWB impulse signal as described with reference to FIG. 1 is only an example, and a method of measuring a vital sign is not limited to the above-described method. In the present disclosure, the device 10 may measure a vital sign of the user 1 through any known method using a UWB impulse signal.

The device 10 may display the measured heart rate. In the embodiment illustrated in FIG. 1, the heart rate of the user 1 is 85 beats per minute (bpm), but is not limited thereto. In an embodiment of the present disclosure, the device 100 may display the measured respiration rate of the user 1.

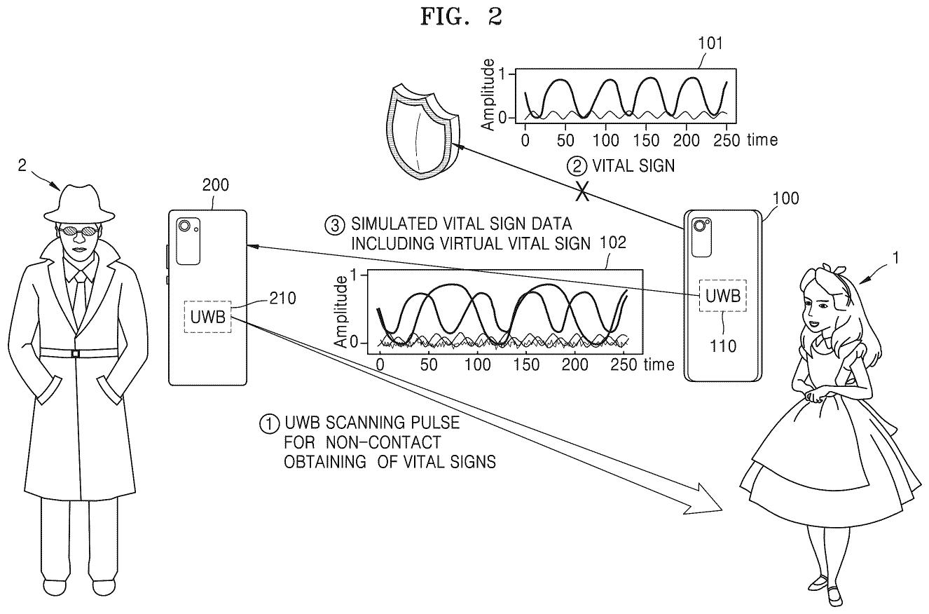

FIG. 2 is a conceptual diagram illustrating an operation in which a device 100 recognizes a UWB scanning pulse transmitted by an external device 200 and outputs simulated vital sign data, according to an embodiment of the present disclosure.

Referring to FIG. 2, the device 100 is a device capable of transmitting and receiving data via UWB communication, and may be, for example, a smart phone or tablet personal computer (PC) including a UWB communication interface (or module) 110. However, the device 100 is not limited thereto, and may also be implemented as a laptop computer, a desktop PC, a smart television (TV), an e-book terminal, a digital broadcast terminal, a personal digital assistant (PDA), a portable multimedia player (PMP), a navigation device, an MP3 player, or a camcorder that includes UWB communication functions. For example, the device 100 may be implemented as a wearable device worn by the user 1 on a part of the body, such as a smartwatch (e.g., Samsung Galaxy Watch, Samsung Fit, etc.) or wireless earphones (e.g., Samsung Galaxy Buds, etc.).

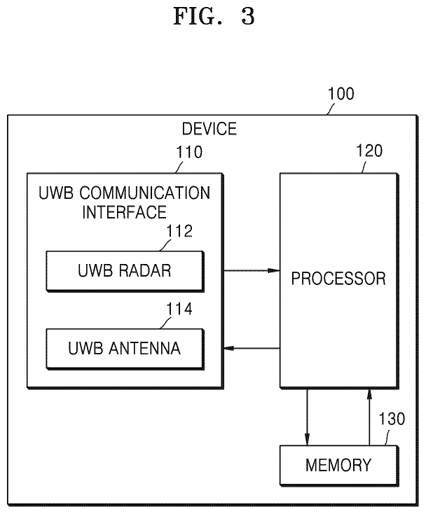

The UWB communication interface 110 is a hardware communication interface or module configured to perform data transmission and reception by using an UWB frequency band between 3.1 gigahertz (GHz) and 10.6 GHz, and may include a UWB radar (112 of FIG. 3) and a UWB antenna (114 of FIG. 3). The external device 200 may include a UWB communication interface 210, and transmit a UWB scanning pulse to the body of the user 1 of the device 100 by using the UWB communication module 210 in order to obtain a vital sign of the user 1 in a non-contact manner (Operation 1). By using the UWB radar 112, the device 100 may detect the UWB scanning pulse transmitted by the external device 200.

When the UWB scanning pulse is detected, the device 100 may block output of a UWB signal component including a vital sign 101 to prevent the vital sign 101 of the user 1 from being transmitted to the external device 200 (Operation (2)). The device 100 may generate a virtual vital sign by simulating a vital sign of a virtual person (a fake person) that does not exist. The ‘virtual vital sign’ may include, for example, at least one of a virtual respiration rate, a virtual heart rate, or a virtual heartbeat interval.

In an embodiment of the present disclosure, the device 100 may generate a virtual vital sign through a simulation based on the vital sign 101 of the user. For example, the device 100 may generate a virtual vital sign by performing a simulation for modulating, processing, or modifying the vital sign 101 of the user.

The device 100 may output simulated vital sign data 102, including the generated virtual vital sign, toward the external device 200 (Operation (3)). The simulated vital sign data 102 may include the vital sign 101 of the user 1 and the generated virtual vital sign. The device 100 may output a UWB signal component including the simulated vital sign data 102 to the external device 200 by using the UWB antenna (114 of FIG. 3). In an embodiment of the present disclosure, the simulated vital sign data 102 may further include context data in addition to the vital sign 101 and the virtual vital sign. The context data is information about an environment or situation surrounding the device 100 and the external device 200, and may include, for example, information about at least one of sound, vibration, light, or atmospheric pressure.

Recently, as illustrated in FIG. 2, a vital sign (e.g., a respiration rate, a heart rate, etc.) of the user 1 of the device 100 may be measured in a non-contact manner by the external device 200, such as a smartphone including a UWB radar. Because non-contact vital sign measurement technology may unintentionally allow a person other than the user 1 of the device 100 (e.g., a user 2 of the external device 200) to measure the vital sign of the user 1 without permission, the user 1 needs to prevent the vital sign 101 from being leaked to the external device 200 in order to protect his or her privacy. In addition, the user 1 may prevent the leakage of the vital sign 101 by outputting a noise signal that causes communication interference to the external device 200 via the UWB communication interface of the device 100. However, the noise signal may cause interference with UWB communication and impair a normal function of the UWB radar, and thus may hinder exchange of legally permitted data.

The present disclosure aims to provide the device 100 that outputs simulated vital sign data in order to protect the vital sign 101 of the user from the external device 200 that attempts to obtain the vital sign 101 of the user in a non-contact manner via a UWB signal, and a an operation method of the device 100.

According to the embodiment illustrated in FIG. 2, when the device 100 detects a UWB scanning pulse for obtaining the vital sign 101 of the user in a non-contact manner by the external device 200, the device 100 generates a virtual vital sign by simulating a vital sign of a virtual person, and outputs the simulated vital sign data including the vital sign 101 of the user and the virtual vital sign via the UWB antenna (114 of FIG. 3), thereby preventing the external device 200 from measuring the vital sign of the user 1. Accordingly, according to an embodiment of the present disclosure, the device 100 may provide technical effects of protecting personal information of the user 1, such as a respiration rate, a heart rate, or a heartbeat interval, from the external device 200, and enhancing security. In addition, according to an embodiment of the present disclosure, the device 100 may output simulated vital sign data that may prevent leakage of the vital sign 101 within a legally permitted range without causing interference with normal UWB communication, and thus may avoid impairing the function of the UWB communication interface 210 of the external device 200.

FIG. 3 is a block diagram illustrating components of the device 100 according to an embodiment of the present disclosure.

According to an embodiment of the present disclosure, the device 100 may be a smartphone or a tablet PC. However, the device 100 is not limited thereto, and may also be implemented as a laptop computer, a desktop PC, a smart TV, an e-book terminal, a digital broadcast terminal, a PDA, a PMP, a navigation device, an MP3 player, a camcorder, a wearable device, or the like.

In an embodiment of the present disclosure, the device 100 may be implemented as an augmented reality device. In the present disclosure, an ‘augmented reality device’ is a device capable of representing augmented reality, and generally includes not only augmented reality glasses in the form of eyeglasses worn on a user's face, but also a head-mounted display (HMD) apparatus or an augmented reality helmet worn on the user's head.

Referring to FIG. 3, the device 100 may include a UWB communication interface 110, at least one processor (also referred to as “the processor”) 120, and memory 130. The UWB communication interface 110, the at least one processor 120, and the memory 130 may be operatively, electrically and/or physically connected to each other.

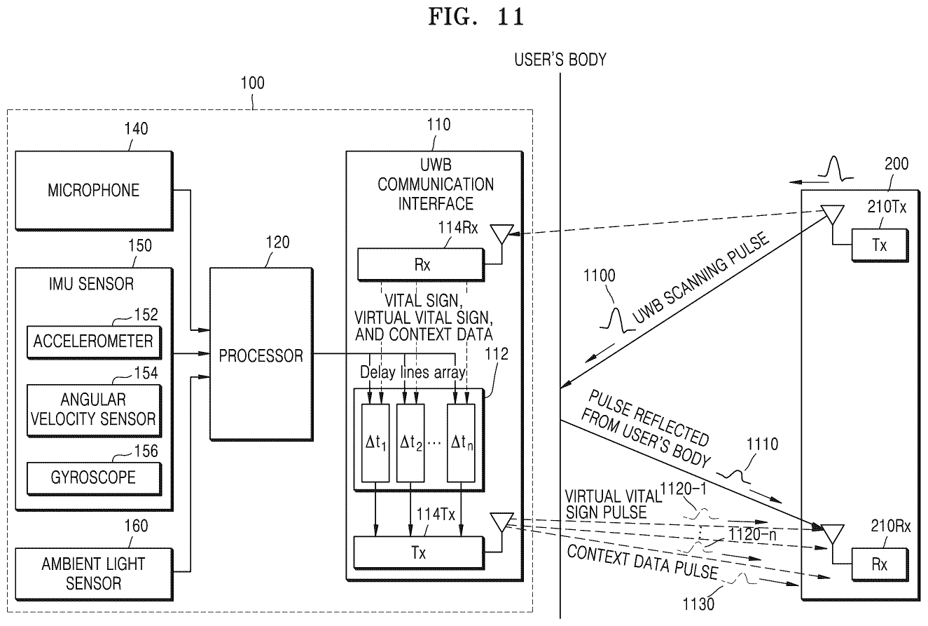

The components shown in FIG. 3 are only in accordance with an embodiment of the present disclosure, and the components included in the device 100 are not limited to those shown in FIG. 3. The device 100 may not include some of the components shown in FIG. 3, and may further include components not shown in FIG. 3. In an embodiment of the present disclosure, the device 100 may further include an inertial measurement unit (IMU) sensor (150 of FIG. 11) that includes an accelerometer, an angular velocity sensor, and a gyroscope and is configured to measure the moving speed, direction, angle, and gravitational acceleration of the device 100. In an embodiment of the present disclosure, the device 100 may further include a microphone (140 of FIG. 11) that obtains a voice uttered by the user or a sound emitted from an external object, and converts the obtained voice or sound into an audio signal. In an embodiment of the present disclosure, the device 100 may further include an ambient light sensor (160 of FIG. 11) that measures illuminance of an external environment. In an embodiment of the present disclosure, the device 100 may be configured as a portable device, and may further include a battery for supplying driving power to the UWB communication interface 110 and the processor 120.

The UWB communication interface 110 is configured as a hardware communication device that performs data transmission and reception by using a UWB frequency band between 3.1 GHz and 10.6 GHz. The UWB communication interface 110 is capable of transmitting and receiving data at speeds up to 500 megabits per second (Mbps). In an embodiment of the present disclosure, the UWB communication interface 110 may include the UWB radar 112 and the UWB antenna 114.

The UWB radar 112 is configured as a communication device that determines and detects the presence, location, distance, speed, or status of an object by emitting radio waves at a UWB frequency toward the object and receiving reflected waves from the object. In an embodiment of the present disclosure, the UWB radar 112 may transmit a UWB impulse signal to a body (e.g., a chest) of the user and receive a signal reflected from the body. The UWB radar 112 may provide the received reflected signal to the processor 120, and the processor 120 may amplify and filter the reflected signal, transform the signal in the time domain into a signal in the frequency domain, and obtain a vital sign such as a user's respiration rate or heart rate by using the transformed signal. In an embodiment of the present disclosure, to obtain a vital sign of the user in a non-contact manner, the UWB radar 112 may detect a UWB scanning pulse transmitted by the external device (200 of FIG. 2).

The UWB antenna 114 is an antenna that transmits a UWB signal component to the outside or receives a UWB signal from the outside. The UWB antenna 114 may include a transmitting antenna (114Tx of FIG. 11) and a receiving antenna (114Rx of FIG. 11).

In an embodiment of the present disclosure, the transmitting antenna 114Tx may include at least one antenna element and transmit a UWB signal component to an external object or external device by using the at least one antenna element. When the transmitting antenna 114Tx includes a plurality of antenna elements, the plurality of antenna elements may be configured as patch antennas, but are not limited thereto.

When an external device is capable of receiving a UWB signal, the UWB antenna 114 may transmit a ranging request message (poll message) via the transmitting antenna 114Tx, and receive, via the receiving antenna 114Rx, a response message received from the external device in response to the ranging request signal. The processor 120 may obtain location information about the external device by using a time of arrival (TOA) or time difference of arrival (TDOA) method that uses a time difference between the ranging request message and the response message. In an embodiment of the present disclosure, the processor 120 may obtain ranging information, which is information about a relative distance between the device 100 and the external device, and angle of arrival (AOA) information, which is direction information of the external device.

In an embodiment of the present disclosure, the UWB antenna 114 may output simulated vital sign data provided by the processor 120 toward the external device. As described above, when the external device is capable of performing UWB communication and a response message is received from the external device, the processor 120 may determine a location of the external device. In this case, the UWB antenna 114 may transmit simulated vital sign data to the external device by using location information of the external device. In an embodiment of the present disclosure, the UWB antenna 114 may transmit, to the external device, the simulated vital sign data as well as context data obtained through the microphone, the IMU sensor, or the ambient light sensor.

The at least one processor 120 individually or collectively may execute one or more instructions of a program stored in the memory 130. The processor 120 may be composed of hardware components that perform arithmetic, logic, and input/output (I/O) operations, and signal processing. For example, the processor 120 may consist of at least one of a CPU, a microprocessor, a graphics processing unit (GPU), application specific integrated circuits (ASICs), digital signal Processors (DSPs), digital signal processing devices (DSPDs), programmable logic devices (PLDs), and field programmable gate arrays (FPGAs), but is not limited thereto. In an embodiment of the present disclosure, when the device 100 is a mobile device such as a smartphone or tablet PC, the processor 120 may be implemented as an application processor.

The processor 120 is shown as an element in FIG. 3, but is not limited thereto. In an embodiment, the processor 120 may be configured as a single processor or a plurality of processors. The processor 130 according to an embodiment of the disclosure may include various processing circuitry and/or multiple processors. For example, as used herein, including the claims, the term “processor” may include various processing circuitry, including at least one processor, wherein one or more of at least one processor, individually and/or collectively in a distributed manner, may be configured to perform various functions described herein. As used herein, when “a processor”, “at least one processor”, and “one or more processors” are described as being configured to perform numerous functions, these terms cover situations, for example and without limitation, in which one processor performs some of recited functions and (an) other processor(s) perform(s) others of the recited functions, and also situations in which a single processor may perform all recited functions. Additionally, the at least one processor may include a combination of processors performing a variety of the recited/disclosed functions, e.g., in a distributed manner. At least one processor may execute program instructions to achieve or perform various functions.

In an embodiment of the present disclosure, the processor 120 may include an artificial intelligence (AI) processor that performs AI training. In this case, the AI processor may generate a virtual vital sign by modulating, processing, or modifying vital sign data of the user by using an AI model. The AI processor may be manufactured in the form of a dedicated hardware chip for AI, or it may be manufactured as a part of an existing general-purpose processor (e.g., a CPU or an application processor) or dedicated graphics processor (e.g., a GPU) and mounted on the processor 120 within the device 100.

The memory 130 may include at least one type of storage medium among, for example, a flash memory-type memory, a hard disk-type memory, a multimedia card micro-type memory, a card-type memory (e.g., a Secure Digital (SD) card or an extreme Digital (XD) memory), random access memory (RAM), static RAM (SRAM), read-only memory (ROM), electrically erasable programmable ROM (EEPROM), PROM, or an optical disc. In an embodiment of the present disclosure, the memory 130 may be implemented as a web storage or a cloud server that is accessible over a network and performs a storage function. In this case, the device 100 may further include a wireless communication interface or module configured to perform data communications over a WiFi or mobile communication network, and may be communicatively coupled to the web storage or the cloud server via the wireless communication interface or module and perform data transmission and reception therewith.

The memory 130 may store instructions or program code for performing, by the device 100, operations of detecting a UWB scanning pulse transmitted by an external device, generating a virtual vital sign by simulating a vital sign of a virtual person in response to the UWB scanning pulse being detected, obtaining simulated vital sign data by combining a vital sign of the user with the virtual vital sign, and outputting the obtained vital sign data. In an embodiment of the present disclosure, the memory 130 may store at least one of instructions, algorithms, data structures, program code, or application programs readable by the processor 120. The instructions, algorithms, data structures, and program code stored in the memory 130 may be implemented in programming or scripting languages such as C, C++, Java, assembler, etc.

In embodiments described below, the processor 120 may be implemented by executing instructions or program code stored in the memory 130.

The processor 120 may obtain a vital sign including at least one of a user's respiration rate, heart rate, or heartbeat interval via the UWB communication interface 110. In an embodiment of the present disclosure, the vital sign of the user may include the respiration rate, heart rate, or heartbeat interval. A specific method by which the device 100 obtains a vital sign of the user in a non-contact manner by using a UWB signal is the same as the method described with reference to FIG. 1, and thus, redundant description is omitted herein. However, the device 100 is not limited to obtaining the vital sign of the user in a non-contact manner by using the UWB signal. In an embodiment of the present disclosure, the device 100 may further include a heart rate sensor or a respiration monitoring sensor, and obtain a vital sign including at least one of a user's respiration rate, heart rate, or heartbeat interval by using the heart rate sensor or the respiration monitoring sensor.

In a case that the processor 120 detects a UWB scanning pulse transmitted by an external device via the UWB radar 112 of the UWB communication interface 110, the processor 120 may generate a virtual vital sign by simulating a vital sign of a virtual person (a fake person). The processor 120 may generate a virtual vital sign by performing a simulation of modulating, processing, or modifying a vital sign of the user. For example, the processor 120 may generate a virtual vital sign via a simulation performed by increasing or decreasing the number of respirations per unit time, or increasing or decreasing the number of heartbeats per unit time. In another example, the processor 120 may generate a virtual vital sign via a simulation performed by increasing or decreasing a heartbeat interval of the user. A virtual vital sign is a vital sign of a virtual person that does not exist, and may include, for example, at least one of a virtual respiration rate, a virtual heart rate, or a virtual heartbeat interval.

The processor 120 may generate a virtual vital sign by performing a simulation using the user's vital sign and context information. The context information refers to information about an environment or situation surrounding the device 100 and the external device. The context information may include, for example, information about at least one of sound, vibration, illumination, or atmospheric pressure. In an embodiment of the present disclosure, the device 100 may further include a microphone, and obtain, by using the microphone, a user's voice or a sound generated by an object in an external environment. In an embodiment of the present disclosure, the device 100 may further include an IMU sensor, and recognize vibrations from the outside or vibration of the device 100 by using the IMU sensor. In an embodiment of the present disclosure, the device 100 may further include an ambient light sensor, and may measure illuminance of an external environment by using the ambient light sensor. The processor 120 may generate a virtual vital sign via a simulation performed by modulating, processing, or modifying the user's vital sign based on the context information. In an embodiment of the present disclosure, the processor 120 may predict how a vital sign changes in response to context information, e.g., in a situation such as sound, vibration, illuminance change, etc., through a trained AI model, and generate a virtual vital sign according to a result of the prediction.

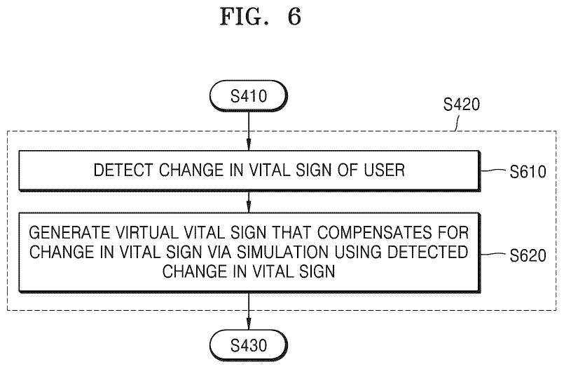

The processor 120 may detect a change in the user's vital sign via the UWB communication interface 110. For example, the user's respiration rate may increase or decrease over time, or the user's heart rate may increase or decrease over time. The processor 120 may obtain information about a rate of change in the vital sign. In an embodiment of the present disclosure, the processor 120 may calculate a rate of increase or decrease of the user's respiration rate or heart rate per unit time. The processor 120 may generate a virtual vital sign that compensates the vital sign through a simulation using the detected change in the vital sign. In an embodiment of the present disclosure, the processor 120 may perform a simulation of decreasing or increasing the virtual vital sign based on a rate of increase or decrease of the vital sign over time. For example, the processor 120 may perform a simulation of decreasing the virtual vital sign by a value equal to a value of a rate of increase of the vital sign over time. In addition, the processor 120 may perform a simulation of increasing the virtual vital sign by a value equal to a value of a rate of decrease of the vital sign over time. A specific method by which the processor 120 generates a virtual vital sign based on the rate of change in a vital sign is described in detail with reference to FIGS. 6 to 8.

The processor 120 may generate a plurality of virtual vital signs having different values from each other by simulating vital signs of a plurality of virtual persons. In an embodiment of the present disclosure, by performing a simulation multiple times, the processor 120 may generate a plurality of virtual respiration rates, a plurality of virtual heart rates, or a plurality of virtual heartbeat intervals.

The processor 120 may obtain simulated vital sign data by combining the generated virtual vital sign with the user's vital sign. The processor 120 may generate a UWB signal component including the simulated vital sign data, and output the UWB signal component by using the UWB antenna 114. In an embodiment of the present disclosure, the processor 120 may also generate a UWB signal component by adding a noise signal (noise) to the vital sign data.

The processor 120 may output the UWB signal component toward the external device. In an embodiment of the present disclosure, in a case that the external device is capable of transmitting and receiving data over a UWB communication network, the processor 120 may obtain location information of the external device based on a response message received from the external device via the receiving antenna of the UWB antenna 114, and transmit the UWB signal component to the external device by using the obtained location information.

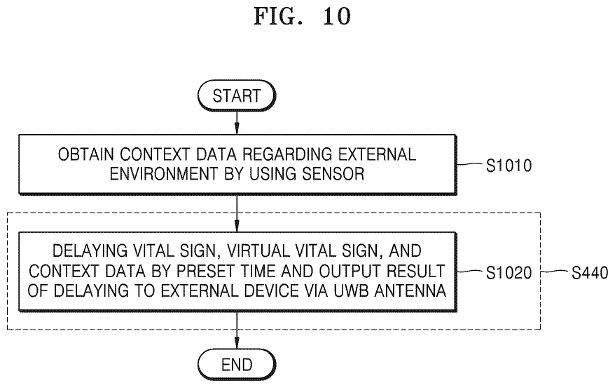

In an embodiment of the present disclosure, the device 100 may include at least one of a microphone, an IMU sensor, and an ambient light sensor, and obtain context data regarding an external environment by using the at least one of the microphone, the IMU sensor, and the ambient light sensor. The context data may include, for example, measurement data regarding at least one of sound, vibration, illumination, or atmospheric pressure. The processor 120 may delay the user's vital sign, virtual vital sign, and context data by a preset time, respectively, and control the UWB antenna 114 to output the delayed user's vital sign, virtual vital sign, and context data to the external device. In an embodiment of the present disclosure, the processor 120 may delay the vital sign, virtual vital sign, and context data by respectively applying different delay times thereto, and control a timing at which the delayed vital sign, the delayed virtual vital sign, and the delayed context data are output through the UWB antenna 114. A specific embodiment in which the processor 120 delays a vital sign, a virtual vital sign, and context data and outputs the delayed vital sign, virtual vital sign, and context data to an external device is described in detail with reference to FIGS. 10 and 11.

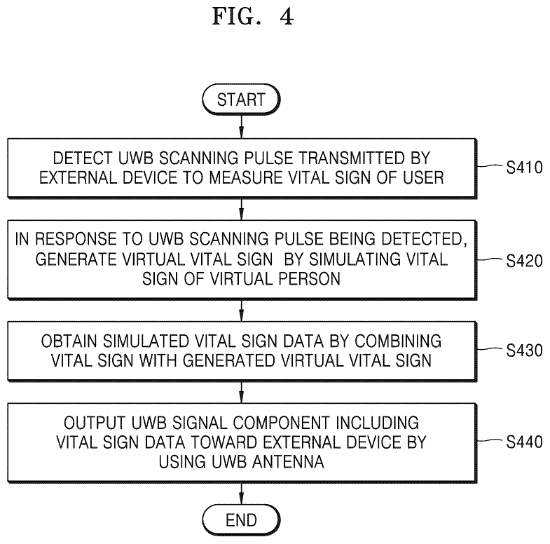

FIG. 4 is a flowchart of an operation method of the device 100, according to an embodiment of the present disclosure.

In operation S410, the device 100 detects a UWB scanning pulse transmitted by an external device to measure a vital sign of the user. The external device may include a UWB communication interface, and transmit a UWB scanning pulse to a body of the user of the device 100 by using the UWB communication interface in order to obtain a vital sign of the user in a non-contact manner. By using the UWB radar (112 of FIG. 3), the device 100 may detect the UWB scanning pulse transmitted by the external device.

In operation S420, in response to the UWB scanning pulse being detected, the device 100 generates a virtual vital sign by simulating a vital sign of a virtual person (or fake person). In an embodiment of the present disclosure, the device 100 may generate a virtual vital sign by performing a simulation of modulating, processing, or modifying the vital sign of the user. For example, the device 100 may generate a virtual vital sign via a simulation performed by increasing or decreasing the number of respirations per unit time, or increasing or decreasing the number of heartbeats per unit time. In another example, the device 100 may generate a virtual vital sign via a simulation performed by increasing or decreasing a heartbeat interval of the user. A virtual vital sign is a vital sign of a virtual person that does not exist, and may include, for example, at least one of a virtual respiration rate, a virtual heart rate, or a virtual heartbeat interval.

In an embodiment of the present disclosure, the device 100 may generate a virtual vital sign by performing a simulation using the vital sign of the user and context information. The context information refers to information about an environment or situation surrounding the device 100 and the external device. The context information may include, for example, information about at least one of sound, vibration, illumination, or atmospheric pressure. The device 100 may generate a virtual vital sign via a simulation performed by modulating, processing, or modifying the vital sign of the user based on the context information. In an embodiment of the present disclosure, the device 100 may predict how a vital sign changes in response to context information, e.g., in a situation such as sound, vibration, illuminance change, etc., through a trained AI model, and generate a virtual vital sign according to a result of the prediction.

In an embodiment of the present disclosure, the device 100 may generate a plurality of virtual vital signs having different values from each other by simulating vital signs of a plurality of virtual persons. By performing a simulation multiple times, the device 100 may generate a plurality of virtual respiration rates, a plurality of virtual heart rates, or a plurality of virtual heartbeat intervals.

In operation S430, the device 100 obtains simulated vital sign data by combining the vital sign with the generated virtual vital sign. In an embodiment of the present disclosure, the device 100 may add a noise signal to the vital sign data composed of a combination of the vital sign and the virtual vital sign.

In operation S440, the device 100 outputs a UWB signal component including the simulated vital sign data toward the external device by using the UWB antenna (114 of FIG. 3). In an embodiment of the present disclosure, when the external device is capable of transmitting and receiving data over a UWB communication network, the device 100 may obtain location information of the external device based on a response message received from the external device via the receiving antenna of the UWB antenna 114, and transmit the UWB signal component to the external device by using the obtained location information.

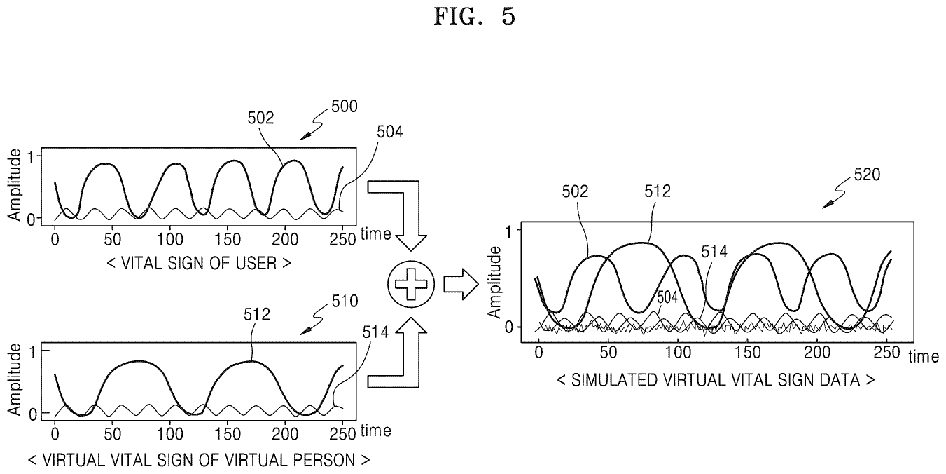

FIG. 5 is a diagram illustrating an operation in which the device 100 obtains simulated vital sign data 520, according to an embodiment of the present disclosure.

Referring to FIG. 5, the device 100 may obtain a vital sign 500 of the user in a non-contact manner by using a UWB signal. In an embodiment of the present disclosure, the vital sign 500 of the user may include a number of respirations (or a respiration rate) 502 and a number of heartbeats (or a heart rate) 504 of the user over time. However, the vital sign 500 of the user is not limited thereto, and the vital sign 500 of the user may further include the user's blood pressure or heartbeat interval.

In an embodiment of the present disclosure, the device 100 may generate an UWB impulse signal by using the UWB radar (112 of FIG. 3), and transmit the UWB impulse signal to a chest of the user by using the UWB antenna (114 of FIG. 3). The transmitted UWB impulse signal is reflected by a body of the user, and the device 100 may receive a reflected UWB impulse signal by using the UWB antenna 114. The device 100 may obtain information about the number of respirations 502 of the user based on a variation in a ToF of the UWB impulse signal according to a mechanical displacement that occurs in the chest and abdominal wall due to the user's respiration. The device 100 may extract a peak interval change value from the received UWB impulse signal, and obtain information about the number of heartbeats 504 of the user by applying filtering and frequency axis transform. A specific method by which the device 100 measures the number of respirations 502 and the number of heartbeats 504 of the user is the same as the method described with reference to FIG. 1, and thus, redundant descriptions are omitted.

However, a method of obtaining a vital sign according to the present disclosure is not limited to the non-contact method described above. In another embodiment of the present disclosure, the device 100 may obtain the vital sign 500 including the number of respirations 502 and the number of heartbeats 504 of the user in a contact manner by using a respiration rate monitoring sensor or a heart rate sensor.

Although it has been described in FIG. 5 that the device 100 obtains a vital sign of the user, the present disclosure is not limited thereto. In an embodiment of the present disclosure, the vital sign 500 of the user may be prestored in a storage space of the memory (130 of FIG. 3) of the device 100.

The device 100 may generate a virtual vital sign 510 by simulating a vital sign of a virtual person (or fake person). The processor (120 of FIG. 3) of the device 100 may generate the virtual vital sign 510 by performing a simulation of modulating, processing, or modifying the vital sign 500 of the user. For example, the processor 120 may generate the virtual vital sign 510 by performing a simulation of increasing or decreasing the number of respirations 502 per unit time in the vital sign 500 of the user, or increasing or decreasing the number of heartbeats 504 per unit time therein. In the embodiment illustrated in FIG. 5, the processor 120 may generate a virtual respiration rate 512 via modulation that is performed by decreasing the number of respirations per unit time by increasing a period of the number of respirations 502 in the vital sign 500. In addition, the processor 120 may generate a virtual heart rate 514 via modulation that is performed by increasing the number of heart beats per unit time by decreasing a period of the number of heartbeats 504 in the vital sign 500.

In an embodiment of the present disclosure, the device 100 may generate the virtual vital sign 510 by performing a simulation using the vital sign 500 of the user and context information. The context information refers to information about an environment or situation surrounding the device 100 and the external device. The context information may include, for example, information about at least one of sound, vibration, illumination, or atmospheric pressure. The processor 120 of the device 100 may generate a virtual vital sign via a simulation performed by modulating, processing, or modifying the vital sign of the user based on the context information. For example, when an external vibration is recognized by the IMU sensor of the device 100 or noise from an external source is obtained via the microphone, the processor 120 may generate the virtual heart rate 514 with an increased number of heartbeats per unit time by performing a simulation of decreasing the period of the number of heartbeats 504 in the vital sign 500 of the user. In another example, when the ambient light sensor of the device 100 recognizes that an illuminance value of an external environment has decreased, the processor 120 may generate the virtual respiration rate 512 with a decreased number of respirations per unit time by performing a simulation of increasing the period of the number of respirations 502 in the vital sign 500 of the user.

The device 100 may obtain the simulated vital sign data 520 by combining the virtual vital sign 510 of the virtual person with the vital sign 500 of the user. The simulated vital sign data 520 may include the real number of respirations 502 and the real number of heartbeats 504 of the user and the virtual respiration rate 512 and the virtual heart rate 514 of the virtual person. In an embodiment of the present disclosure, the device 100 may obtain the vital sign data 520 by adding a noise signal in addition to the vital sign 500 and the virtual vital sign 510.

Although only one virtual vital sign 510 including the one virtual respiration rate 512 and the one virtual heart rate 514 is illustrated in FIG. 5, an embodiment of the present disclosure is not limited thereto. In an embodiment of the present disclosure, the virtual vital sign 510 may be two or more virtual vital signs, and in this case, the simulated vital sign data 520 may include the vital sign 500 of the user and the plurality of virtual vital signs 510.

FIG. 6 is a flowchart of a method, performed by the device 100, of generating a virtual vital sign based on a change in the vital sign of the user, according to an embodiment of the present disclosure.

Operations S610 and S620 illustrated in FIG. 6 are detailed operations of operation S420 illustrated in FIG. 4. Operation S610 of FIG. 6 may be performed after operation S410 illustrated in FIG. 4 is performed. Operation S430 illustrated in FIG. 4 may be performed after operation S620 of FIG. 6 is performed.

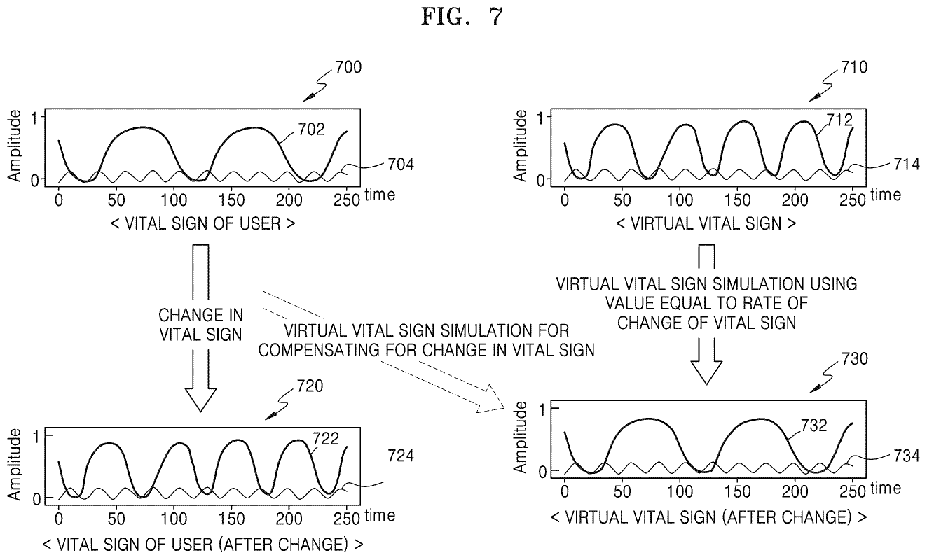

FIG. 7 is a flowchart of a method, performed by the device 100, of generating a virtual vital sign 710 by performing a simulation for compensating for a change in a vital sign 700 of the user, according to an embodiment of the present disclosure.

Hereinafter, an operation in which the device 100 generates the virtual vital sign 710 according to a change in the vital sign 700 of the user is described with reference to FIGS. 6 and 7 together.

In operation S610 of FIG. 6, the device 100 detects a change in the vital sign of the user. Referring to FIG. 7 in conjunction with FIG. 6, the vital sign 700 of the user may include a respiration rate 702 and a heart rate 704 over time. Referring to a vital sign 720 of the user after a change in the vital sign occurs, a respiration rate 722 has a reduced period and an increased number of respirations per unit time compared to the respiration rate 702 before the change occurs. Furthermore, a heart rate 724 of the user after the change in the vital sign occurs has no substantial difference compared to the heart rate 704 before the change occurs, but a period thereof has shifted in the time domain. Unlike in FIG. 7, the heart rate 724 may have a reduced period and an increased number of heartbeats per unit time compared to the heart rate 704 before the change occurs.

The processor (120 of FIG. 3) of the device 100 may obtain information about a rate of change in the vital sign based on a difference between the vital sign 700 of the user generated before the change and the vital sign 720 of the user generated after the change. In an embodiment of the present disclosure, the processor 120 may obtain information about a rate of change of a period of the respiration rate 702 or heart rate 704 of the user in the time domain, and calculate a rate of increase or decrease of the respiration rate 702 or heart rate 704 per unit time based on the obtained rate of change of the period.

Referring to operation S620 of FIG. 6, the device 100 generates a virtual vital sign that compensates for the change in the vital sign via a simulation using the detected change in the vital sign. In an embodiment of the present disclosure, the device 100 may calculate a rate of the detected increase or decrease of the vital sign and perform a simulation of decreasing or increasing the virtual vital sign based on the rate of increase or decrease of the vital sign over time. Referring to FIG. 7 in conjunction with FIG. 6, in order to offset the change in the vital sign 700, the device 100 may perform a simulation of decreasing or increasing the virtual vital sign 710 by a value equal to a value of the rate of increase or decrease of the vital sign 700. The virtual vital sign 710 before the change in the vital sign 700 may include a virtual respiration rate 712 and a virtual heart rate 714. The processor 120 of the device 100 may decrease or increase the virtual respiration rate 712 or the virtual heart rate 714 in the virtual vital sign 710 per unit time by a value equal to the rate of increase or decrease of the respiration rate 702 or heart rate 704 per unit time, which is calculated in operation S610. Through the simulation, the processor 120 may obtain a virtual vital sign 730 generated after the change.

In the embodiment illustrated in FIG. 7, because the period of the respiration rate 702 in the vital sign 700 has decreased and the respiration rate 702 per unit time has increased, the processor 120 may calculate a rate of increase of the respiration rate per unit time based on a difference between the period of the respiration rate 722 after the change in the vital sign and the period of the respiration rate 702 before the change, and may decrease the virtual respiration rate 712 per unit time in the virtual vital sign 710 by a value equal to the rate of increase of the respiration rate per unit time. In accordance with the decrease in the virtual respiration rate 712 per unit time, the period of a virtual respiration rate 732 in the time domain after the change may increase. Similarly, in a case that the heart rate 704 per unit time increases due to the change in the vital sign 700, the processor 120 may calculate a rate of increase of the heart rate 704 per unit time and decrease the virtual heart rate 714 per unit time in the virtual vital sign 710 by a value equal to the calculated rate of increase. In accordance with the decrease in the virtual heart rate 714 per unit time, the period of a virtual heart rate 734 in the time domain after the change may increase.

As an example contrary to the example of FIG. 7, in a case that the period of the respiration rate 702 increases and the respiration rate 702 per unit time decreases due to the change in the vital sign 700, the processor 120 may calculate a rate of decrease of the respiration rate per unit time based on a difference between the period of the respiration rate 722 after the change in the vital sign and the period of the respiration rate 702 before the change, and may increase the virtual respiration rate 712 per unit time in the virtual vital sign 710 by a value equal to the calculated rate of decrease.

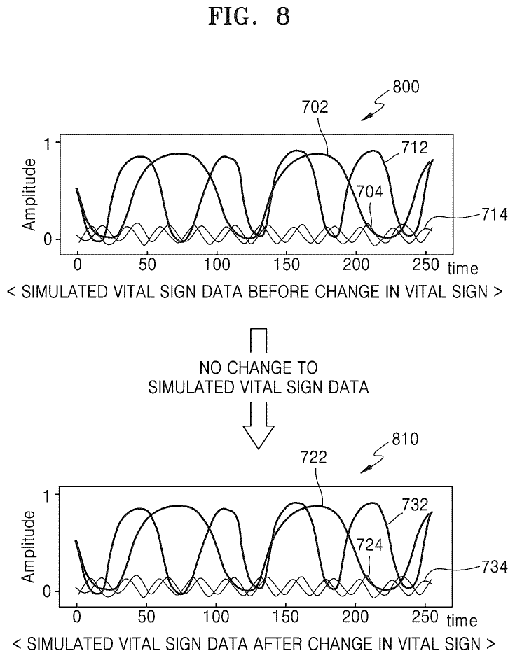

According to the embodiment illustrated in FIGS. 6 and 7, in a case that a change in the vital sign 700 of the user is detected, the device 100 may perform a simulation of decreasing or increasing the virtual respiration rate 712 or virtual heart rate 714 in the virtual vital sign 710 by a value equal to the rate of increase or decrease of the respiration rate 702 or heart rate 704 per unit time included in the vital sign 700 in order to offset a value of the change of the vital sign 700. Therefore, according to an embodiment of the present disclosure, the device 100 has a technical effect whereby it may be difficult for an external device to detect a change in the vital sign even if the change in the vital sign occurs by combining together the vital sign 720 and the virtual vital sign 730 generated after the change. This is described in detail with reference to FIG. 8.

FIG. 8 is a diagram illustrating simulated vital sign data before and after a change in a vital sign occurs.

Referring to FIG. 8, the device 100 may obtain simulated vital sign data 800 by combining together the vital sign (700 of FIG. 7) and the virtual vital sign (710 of FIG. 7) generated before a change in a vital sign occurs. The simulated vital sign data 800 may include the respiration rate 702 and the heart rate 704 in the vital sign 700 of the user and the virtual respiration rate 712 and the virtual heart rate 714 in the virtual vital sign 710. In an embodiment of the present disclosure, the simulated vital sign data 800 may further include a noise signal.

After the change in the vital sign occurs, the device 100 may obtain simulated vital sign data 810 by combining the vital sign (720 of FIG. 7) generated after the change with the virtual vital sign (730 of FIG. 7) generated after the change. The simulated vital sign data 810 after the change may include the respiration rate 722 and the heart rate 724 in the vital sign 720 of the user after the change, and the virtual respiration rate 732 and the virtual heart rate 734 in the virtual vital sign 730 after the change.

As described with reference to FIGS. 6 and 7, the virtual vital sign 730 after the change is generated by performing a simulation of decreasing or increasing the virtual vital sign 710 by a value equal to a rate of increase or decrease between the vital sign 700 before the change and the vital sign 720 after the change, and thus, the vital sign data 800 which is a combination of the vital sign 700 and the virtual vital sign 710 generated before the change may be composed of substantially the same signals as the vital sign data 810 which is a combination of the vital sign 720 after the change and the virtual vital sign 730 generated after the change. Referring to the embodiments illustrated in FIGS. 6 to 8, the device 100 may change the virtual vital signs 710 and 730 to compensate for or offset change values even if changes occur in the vital signs 700 and 720 of the user, so that the simulated vital sign data 800 before the change may be maintained the same as the simulated vital sign data 810 after the change. Thus, according to an embodiment of the present disclosure, the device 100 provides a technical effect of preventing the vital signs 700 and 720 of the user from being leaked by an external device, protecting personal information of the user, and enhancing security.

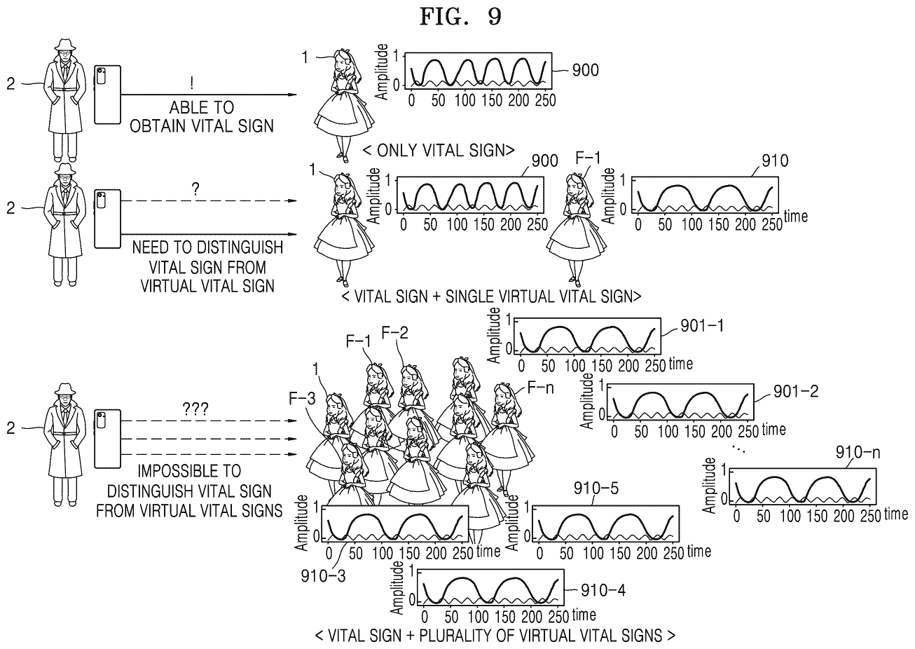

FIG. 9 is a diagram illustrating whether an external device is able to distinguish a vital sign 900 of a user according to the number of virtual vital signs generated by the device 100, according to an embodiment of the present disclosure.

Referring to FIG. 9, when there is only the vital sign 900 of a user 1, the external device 200 may obtain the vital sign 900 of the user in a non-contact manner by using a UWB scanning pulse. In a case that the user 1 does not grant the user 2 of the external device 200 the authority to obtain the vital sign 900, the vital sign 900 may be leaked to the outside, resulting in a problem in that the privacy of the user 1 is violated.

In a case that there is vital sign data including the vital sign 900 of the user 1 and a virtual vital sign 910 of a virtual person F-1, the external device 200 may obtain the vital sign data in a non-contact manner by using a UWB scanning pulse. The external device 200 may attempt to distinguish the vital sign 900 of the user 1 from the virtual vital sign 910 of the virtual person F-1 by using signal processing, filtering, or an AI model. However, it takes processing time for the external device 200 to distinguish the vital sign 900 from the virtual vital sign 910, and it is difficult to accurately extract only the vital sign 900 from the vital sign data.

In a case that there is vital sign data including the vital sign 900 of the user 1 and a plurality of virtual vital signs 910-1 to 910-n of a plurality of virtual persons F-1 to F-n, the external device 200 may obtain the vital sign data in a non-contact manner by using a UWB scanning pulse. The external device 200 may attempt to extract the vital sign 900 of the user 1 from the vital sign data including the plurality of virtual vital signs 910-1 to 910-n, but it is impossible for the external device 200 to distinguish the vital sign 900 from the plurality of pieces of vital sign data.

As illustrated in FIG. 9, in a case that the vital sign data includes the plurality of virtual vital signs 910-1 to 910-n, there is a lower possibility that the vital sign 900 of the user 1 will be leaked by the external device 200 than in a case that the vital sign data includes only the single virtual vital sign 910. According to an embodiment of the present disclosure, by generating the plurality of virtual vital signs 910-1 to 910-n and obtaining the vital sign data by combining the vital sign of the user 1 with the plurality of virtual vital signs 910-1 to 910-n, the device 100 may protect personal information of the user 1 from the outside and enhance security.

FIG. 10 is a flowchart of a method, performed by the device 100, of delaying a vital sign, a virtual vital sign, and context data and outputting the delayed vital sign, the delayed virtual vital sign, and the delayed context data to an external device, according to an embodiment of the present disclosure.

FIG. 11 is a diagram illustrating an operation in which the device 100 delays a vital sign, a virtual vital sign, and context data and outputs the delayed vital sign, the delayed virtual vital sign, and the delayed context data to an external device 200, according to an embodiment of the present disclosure.

Hereinafter, an operation in which the device 100 delays a vital sign, a virtual vital sign, and context data and outputs the delayed vital sign, the delayed virtual vital sign, and the delayed context data to the external device 200 is described with reference to FIG. 10 and FIG. 11 together.

Referring to operation S1010 of FIG. 10, the device 100 obtains context data regarding an external environment by using a sensor. As used herein, ‘context data’ is information about an environment or situation surrounding the device 100 and the external device (200 of FIG. 11), and may include, for example, information about at least one of sound, vibration, illumination, or atmospheric pressure. Referring to FIG. 11 in conjunction with FIG. 10, the device 100 may include the microphone 140, the IMU sensor 150, and the ambient light sensor 160, as well as the UWB communication interface 110 and the processor 120.

The microphone 140 is a device configured to obtain a voice or other sounds from a user or an external source, and convert the obtained voice or other sounds into an audio signal. In an embodiment of the present disclosure, the microphone 140 may be configured as a microphone array consisting of a plurality of microphone elements, a directional microphone, or a multi-pattern microphone. The microphone 140 may provide an audio signal obtained from a user or an external object to the processor 120.

The IMU sensor 150 is a sensor configured to measure the moving the speed, direction, angle, and gravitational acceleration of movement of the device 100. The IMU sensor 150 may include an accelerometer 152, an angular velocity sensor 154, and a gyroscope 156. In an embodiment of the present disclosure, the IMU sensor 150 may measure accelerations in a X-axis direction, a Y-axis direction, and a Z-axis direction by using the three-axis accelerometer 152, and may measure roll, pitch, and yaw angular velocities by using the three-axis angular velocity sensor 154. In an embodiment of the present disclosure, the IMU sensor 150 may measure an angular velocity by using the gyroscope 156, and detect a direction of gravity based on the measured angular velocity. The IMU sensor 150 may provide the processor 120 with the measured three-axis accelerations and three-axis angular velocities, or information about the direction of gravity.

The ambient light sensor 160 is a sensor configured to measure the illuminance of an external environment of the device 100. The ambient light sensor 160 may provide the measured illuminance value to the processor 120.

Referring to operation S1020 of FIG. 10, the device 100 delays a vital sign, a virtual vital sign, and context data by a preset time and outputs the delayed vital sign, the delayed virtual vital sign, and the delayed context data to the external device via a UWB antenna. Operation S1020 illustrated in FIG. 10 is a detailed operation of operation S440 illustrated in FIG. 4. Referring to FIG. 11 in conjunction with FIG. 10, the processor 120 of the device 100 may delay a vital sign and a virtual vital sign, as well as context data obtained from the microphone 140, the IMU sensor 150, and the ambient light sensor 160, and output a result of the delaying to the external device 200 via the transmitting antenna 114Tx of the UWB communication interface 110. The context data may include, for example, at least one of an audio signal obtained from the microphone 140, acceleration values, angular velocity values, and gravity direction information obtained from the IMU sensor 150, and an illuminance value obtained from the ambient light sensor 160. However, the present disclosure is not limited thereto, and the device 100 may further include any known type of sensor capable of sensing or measuring from an external environment, and the context data may include measurement value data obtained through the sensors.

The UWB communication interface 110 may include the UWB radar 112, the receiving antenna 114Rx, and the transmitting antenna 114Tx. The receiving antenna 114Rx may receive a UWB scanning pulse 1100 transmitted by a transmitting antenna 210Tx of the external device 200, and provide information about the received UWB scanning pulse 1100 to the UWB radar 112. The UWB radar 112 may provide the processor 120 with a signal indicating that the UWB scanning pulse 1100 has been detected by the external device 200. The UWB radar 112 may include a delay lines array that applies different delay times Δt₁, Δt₂, . . . , and Δtₙ respectively to the vital sign, the virtual vital sign, and the context data received from the processor 120. In response to the UWB scanning pulse 1100 being detected, the processor 120 may control the delay lines array of the UWB radar 112 to delay the vital sign, the virtual vital sign, and the context data by respectively applying the different delay times Δt₁, Δt₂, . . . , and Δtₙ to the vital sign, the virtual vital sign, and the context data. The processor 120 may output the vital sign, the virtual vital sign, and the context data respectively delayed with the different delay times Δt₁, Δt₂, . . . , and Δtₙ to the external device 200 via the transmitting antenna 114Tx. In an embodiment of the present disclosure, the different delay times Δt₁, Δt₂, . . . , and Δtₙ have irregular intervals, and the vital sign, the virtual vital sign, and the context data may be delayed by randomly applying the different delay times Δt₁, Δt₂, . . . , and Δtₙ thereto. In the embodiment illustrated in FIG. 11, the transmitting antenna 114Tx may transmit a plurality of virtual vital sign pulses 1120-1 to 1120-n and a context data pulse 1130 to the external device 200 by applying different delay times thereto according to control by the processor 120.

The external device 200 may receive, via a receiving antenna 210Rx, a pulse 1110 reflected from the user's body as well as the plurality of virtual vital sign pulses 1120-1 to 1120-n and the context data pulse 1130 that have been delayed.

According to the embodiments illustrated in FIGS. 10 and 11, the device 100 delays the vital sign, the virtual vital sign, and the context data by respectively applying the different delay times Δt₁, Δt₂, . . . , and Δtₙ to the vital sign, the virtual vital sign, and the context data by using the delay lines array of the UWB radar 112, and outputs the delayed pulses via the transmitting antenna 114Tx, thereby preventing the external device 200 from accurately extracting the user's vital sign from the pulses received via the receiving antenna 210Rx. Thus, according to an embodiment of the present disclosure, the device 100 provides a technical effect of preventing the user's vital sign from being leaked by the external device 200, protecting personal information of the user, and enhancing security.

The present disclosure provides a device (100) for outputting a simulated vital sign. According to an embodiment of the present disclosure, the device (100) may include a UWB communication interface 110 including a UWB radar 112 and a UWB antenna 114 and configured to detect a UWB scanning pulse transmitted by an external device 200 to measure a vital sign of a user in a non-contact manner, memory 130 storing at least one instruction, and at least one processor 120 configured to execute the at least one instruction. The at least one processor 120 may be configured to, in response to the UWB scanning pulse being detected by the UWB communication interface, generate a virtual vital sign by simulating a vital sign of a virtual person. The at least one processor 120 may be configured to obtain simulated vital sign data by combining the vital sign with the virtual vital sign. The at least one processor 120 may be configured to control the UWB antenna 114 to output a UWB signal component including the vital sign data toward the external device 200.

In an embodiment of the present disclosure, the at least one processor 120 may be configured to generate a virtual vital sign by performing a simulation of modulating, processing, or modifying the vital sign.

In an embodiment of the present disclosure, the at least one processor 120 may be configured to generate a virtual vital sign by performing a simulation using the vital sign and context information.

In an embodiment of the present disclosure, the context information is information about an environment or situation surrounding the device 100 and the external device 200, and may include information about at least one of sound, vibration, illumination, or atmospheric pressure.

In an embodiment of the present disclosure, the at least one processor 120 may be configured to detect a change in the vital sign and generate a virtual vital sign that compensates for the change in the vital sign through a simulation using the detected change in the vital sign.

In an embodiment of the present disclosure, the at least one processor 120 may be configured to perform a simulation of decreasing or increasing the virtual vital sign by a value equal to a value of a rate of increase or decrease of the vital sign per unit time.

In an embodiment of the present disclosure, the at least one processor 120 may be configured to generate a plurality of virtual vital signs having different values from each other by simulating vital signs of a plurality of virtual persons. The simulated vital sign data may include the vital sign and the plurality of virtual vital signs.

In an embodiment of the present disclosure, the device 100 may further include a microphone 140 configured to obtain a voice of the user or a sound from an external object, an IMU sensor 150 configured to measure at least one of an acceleration, an angular velocity, and a gravity direction, and an ambient light sensor 160 configured to measure illuminance of an external environment. The at least one processor 120 may be configured to obtain context data about an external environment from at least one of the microphone 140, the IMU sensor 150, and the ambient light sensor 160, and delay, by a preset time, the vital sign, the virtual vital sign, and the context data. The at least one processor 120 may be configured to control the UWB antenna 114 to output the delayed vital sign, the delayed virtual vital sign, and the delayed context data to the external device 200.

In an embodiment of the present disclosure, the at least one processor 120 may be configured to, by delaying the vital sign, the virtual vital sign, and the context data by respectively applying different delay times to the vital sign, the virtual vital sign, and the context data, control a timing at which the delayed vital sign, the delayed virtual vital sign, and the delayed context data are output through the UWB antenna 114.

In an embodiment of the present disclosure, the at least one processor 120 may be configured to obtain a UWB signal component by adding a noise signal to the vital sign data. The at least one processor 120 may be configured to control the UWB antenna 114 to output the generated UWB signal component toward the external device 200.

The present disclosure provides a method, performed by a device 100, of outputting a simulated vital sign. According to an embodiment of the present disclosure, the method performed by the device 100 may include detecting a UWB scanning pulse transmitted by an external device 200 to measure a vital sign of a user in a non-contact manner (S410). The method may include, in response to the UWB scanning pulse being detected, generating a virtual vital sign by simulating a vital sign of a virtual person (S420). The method may include obtaining simulated vital sign data by combining the vital sign with the generated virtual vital sign (S430). The method may include outputting a UWB signal component including the vital sign data toward the external device 200 by using a UWB antenna (S440).

In an embodiment of the present disclosure, in the generating of the virtual vital sign (S420), the device 100 may be configured to generate a virtual vital sign by performing a simulation of modulating, processing, or modifying the vital sign.

In an embodiment of the present disclosure, in the generating of the virtual vital sign (S420), the device 100 may be configured to generate a virtual vital sign by performing a simulation using the vital sign and context information.

In an embodiment of the present disclosure, the context information is information about an environment or situation surrounding the device 100 and the external device 200, and may include information about at least one of sound, vibration, illumination, or atmospheric pressure.

In an embodiment of the present disclosure, the generating of the virtual vital sign (S420) may include detecting a change in the vital sign (S610), and generating the virtual vital sign that compensates for the change in the vital sign through a simulation using the detected change in the vital sign (S620).

In an embodiment of the present disclosure, in the generating of the virtual vital sign (S420), the device 100 may be configured to generate a plurality of virtual vital signs having different values from each other by simulating vital signs of a plurality of virtual persons. The simulated vital sign data may include the vital sign and the plurality of virtual vital signs.

In an embodiment of the present disclosure, the method may further include obtaining context data about an external environment by using a sensor (S1010). The outputting of the UWB signal component (S440) may include delaying, by a preset time, the vital sign, the virtual vital sign, and the context data, and outputting the delayed vital sign, the delayed virtual vital sign, and the delayed context data to the external device 200 via the UWB antenna (S1020).

In an embodiment of the present disclosure, the outputting of the UWB signal component (S440) may include, by delaying the vital sign, the virtual vital sign, and the context data by respectively applying different delay times to the vital sign, the virtual vital sign, and the context data, controlling a timing at which the delayed vital sign, the delayed virtual vital sign, and the delayed context data are output through the UWB antenna.

In an embodiment of the present disclosure, the outputting of the vital sign data toward the external device 200 (S440) may include UWB signal component (S440) may include obtaining a UWB signal component by adding a noise signal to the vital sign data, and output the generated UWB signal component toward the external device 200 via the UWB antenna 114.

The present disclosure provides a computer program product including a computer-readable storage medium. In an embodiment of the present disclosure, the storage medium may include instructions that are readable by the device 100 to detect a UWB scanning pulse transmitted by an external device 200 to measure a vital sign of a user in a non-contact manner, in response to the UWB scanning pulse being detected, generate a virtual vital sign by simulating a vital sign of a virtual person, obtain simulated vital sign data by combining the vital sign with the generated virtual vital sign, and output a UWB signal component including the vital sign data toward the external device 200 by using the UWB antenna 114.

A program executed by the device 100 described in this specification may be implemented as a hardware component, a software component, and/or a combination of the hardware component and the software component. The program may be executed by any system capable of executing computer-readable instructions.

Software may include a computer program, a piece of code, an instruction, or a combination of one or more thereof, and configure a processing device to operate as desired or instruct the processing device independently or collectively.

The software may be implemented as a computer program including instructions stored in computer-readable storage media. Examples of the computer-readable recording media include magnetic storage media (e.g., ROM, RAM, floppy disks, hard disks, etc.), optical recording media (e.g., compact disc (CD)-ROM and a digital versatile disc (DVD)), etc. The computer-readable recording media may be distributed over computer systems connected through a network so that computer-readable code may be stored and executed in a distributed manner. The media may be readable by a computer, stored in a memory, and executed by a processor.

A computer-readable storage medium may be provided in the form of a non-transitory storage medium. In this regard, the term ‘non-transitory’ only means that the storage medium does not include a signal and is a tangible device, and the term does not differentiate between where data is semi-permanently stored in the storage medium and where the data is temporarily stored in the storage medium. For example, the ‘non-transitory storage medium’ may include a buffer in which data is temporarily stored.

Furthermore, programs according to embodiments of the present disclosure may be included in a computer program product when provided. The computer program product may be traded, as a product, between a seller and a buyer.

The computer program product may include a software program and a computer-readable storage medium having stored thereon the software program. For example, the computer program product may include a product (e.g., a downloadable application) in the form of a software program electronically distributed by a manufacturer of the device 100 or through an electronic market (e.g., Samsung Galaxy Store™). For such electronic distribution, at least a part of the software program may be stored in the storage medium or may be temporarily generated. In this case, the storage medium may be a storage medium of a server of a manufacturer of the device 100, a server of the electronic market, or a relay server for temporarily storing the software program.

In a system including the device 100 and/or a server, the computer program product may include a storage medium of the server or a storage medium of the device 100. Alternatively, in a case where there is a third device communicatively connected to the device 100 (e.g., when the device 100 is a smartphone, the third device is a wearable device), the computer program product may include a storage medium of the third device. Alternatively, the computer program product may include a software program itself that is transmitted from the device 100 to the third device or that is transmitted from the third device to the device 100.

In this case, one of the device 100 and the third device may execute the computer program product to perform methods according to disclosed embodiments. Alternatively, two or more of the device 100, the server, and the third device may execute the computer program product to perform the methods according to the disclosed embodiments in a distributed manner.

For example, the device 100 may execute the computer program product stored in the memory (130 of FIG. 3) to control another electronic device (e.g., a wearable device) communicatively connected to the device 100 to perform the methods according to the disclosed embodiments.

In another example, the third device may execute the computer program product to control an electronic device communicatively connected to the third device to perform the methods according to the disclosed embodiments.

In a case where the third device executes the computer program product, the third device may download the computer program product from the device 100 and execute the downloaded computer program product. Alternatively, the third device may execute the computer program product that is pre-loaded therein to perform the methods according to the disclosed embodiments.

While the embodiments have been described above with reference to the drawings, it will be understood by those of ordinary skill in the art that various modifications and changes in form and details may be made from the above descriptions. For example, adequate effects may be achieved even when the above-described techniques are performed in a different order than that described above, and/or the aforementioned components such as computer systems or modules are coupled or combined in different forms and modes than those described above or are replaced or supplemented by other components or their equivalents.

What is claimed is:

1. A device for outputting a simulated vital sign, the device comprising:
an ultra-wideband (UWB) communication interface comprising a UWB radar and a UWB antenna, the UWB communication interface being configured to detect a UWB scanning pulse transmitted by an external device to measure a vital sign of a user in a non-contact manner;
at least one processor including processing circuitry; and
memory storing one or more instructions;
wherein the one or more instructions are configured to, when executed by the at least one processor individually or collectively, cause the device to:
based on the UWB scanning pulse being detected by the UWB communication interface, generate a virtual vital sign by simulating a vital sign of a virtual person,
obtain simulated vital sign data by combining the vital sign with the generated virtual vital sign, and
output, through the UWB antenna, a UWB signal component comprising the vital sign data toward the external device.

2. The device of claim 1, wherein the one or more instructions are configured to, when executed by the at least one processor individually or collectively, further cause the device to generate the virtual vital sign by performing a simulation using the vital sign and context information.

3. The device of claim 2, wherein the context information comprises information about at least one of sound, vibration, illumination, or atmospheric pressure of an environment or situation surrounding the device and the external device.

4. The device of claim 1, wherein the one or more instructions are configured to, when executed by the at least one processor individually or collectively, further cause the device to:
detect a change in the vital sign, and
generate the virtual vital sign that compensates for the change in the vital sign through a simulation using the detected change in the vital sign.

5. The device of claim 1, further comprising:
a microphone configured to obtain a voice of the user or a sound from an external object;
an inertial measurement unit (IMU) sensor configured to measure at least one of an acceleration, an angular velocity, or a gravity direction; and
an ambient light sensor configured to measure illuminance of an external environment,
wherein the one or more instructions are configured to, when executed by the at least one processor individually or collectively, further cause the device to:
obtain context data about the external environment from at least one of the microphone (140), the IMU sensor, and the ambient light sensor,
delay, by a preset time, the vital sign, the virtual vital sign, and the context data, and
output, through the UWB antenna, the delayed vital sign, the delayed virtual vital sign, and the delayed context data to the external device.

6. The device of claim 5, wherein the one or more instructions are configured to, when executed by the at least one processor individually or collectively, further cause the device to, by delaying the vital sign, the virtual vital sign, and the context data by respectively applying different delay times to the vital sign, the virtual vital sign, and the context data, control a timing at which the delayed vital sign, the delayed virtual vital sign, and the delayed context data are output through the UWB antenna.

7. The device of claim 1, wherein the one or more instructions are configured to, when executed by the at least one processor individually or collectively, further cause the device to:
obtain a UWB signal component by adding a noise signal to the vital sign data, and
output, through the UWB antenna, the generated UWB signal component toward the external device.

8. A method, performed by a device, of outputting a simulated vital sign, the method comprising:
detecting an ultra-wideband (UWB) scanning pulse transmitted by an external device to measure a vital sign of a user in a non-contact manner;
based on the UWB scanning pulse being detected, generating a virtual vital sign by simulating a vital sign of a virtual person;
obtaining simulated vital sign data by combining the vital sign with the generated virtual vital sign; and
outputting, through a UWB antenna of the device, a UWB signal component comprising the vital sign data toward the external device.

9. The method of claim 8, wherein the generating of the virtual vital sign comprises generating the virtual vital sign by performing a simulation using the vital sign and context information.

10. The method of claim 9, wherein the context information comprises information about at least one of sound, vibration, illumination, or atmospheric pressure of an environment or situation surrounding the device and the external device.

11. The method of claim 8, wherein the generating the virtual vital sign comprises:
detecting a change in the vital sign; and
generating the virtual vital sign that compensates for the change in the vital sign through a simulation using the detected change in the vital sign.

12. The method of claim 8, further comprising:
obtaining context data about an external environment by using a sensor,
wherein the outputting the UWB signal component comprises delaying, by a preset time, the vital sign, the virtual vital sign, and the context data and outputting the delayed vital sign, the delayed virtual vital sign, and the delayed context data to the external device through the UWB antenna.

13. The method of claim 12, wherein the outputting the UWB signal component comprises, by delaying the vital sign, the virtual vital sign, and the context data by respectively applying different delay times to the vital sign, the virtual vital sign, and the context data, controlling a timing at which the delayed vital sign, the delayed virtual vital sign, and the delayed context data are output through the UWB antenna.

14. The method of claim 8, wherein the outputting the vital sign data toward the external device comprises:
obtaining a UWB signal component by adding a noise signal to the vital sign data; and
outputting the generated UWB signal component toward the external device via the UWB antenna.

15. A computer program product comprising a non-transitory computer-readable storage medium storing instructions that are executed by at least one processor of a device to perform a method of outputting a simulated vital sign, the method comprising:
detecting an ultra-wideband (UWB) scanning pulse transmitted by an external device to measure a vital sign of a user in a non-contact manner;
based on the UWB scanning pulse being detected, generating a virtual vital sign by simulating a vital sign of a virtual person;
obtaining simulated vital sign data by combining the vital sign with the generated virtual vital sign; and
outputting, through a UWB antenna of the device, a UWB signal component comprising the vital sign data toward the external device.
